(19) United States
(12) Patent Application Publication
(10) Pub. No.: US 2026/0165588 A1
(43) Pub. Date: Jun. 18, 2026

(54) METHODS AND SYSTEMS FOR GATHERING PHYSIOMETRIC DATA FROM A DENTAL SCAN

(71) Applicant: Get-Grin Inc., Airmont, NY (US)

(72) Inventors: Alon Luis LIPNIK, Tel Aviv (IL); Yarden EILAT-BLOCH, Haifa (IL); Carmi RAZ, Gizo (IL)

(21) Appl. No.: 19/531,570

(22) Filed: Feb. 5, 2026

Related U.S. Application Data
(63) Continuation of application No. PCT/US2024/041133, filed on Aug. 6, 2024.
(60) Provisional application No. 63/530,980, filed on Aug. 6, 2023.

(51) Int. Cl. A61B 5/00; A61B 5/024; A61C 9/00

(52) U.S. Cl. CPC A61B 5/0088; A61B 5/02416; A61C 9/0053

(57) ABSTRACT

Described herein are systems and methods for gathering physiometric data from a dental scan of a subject.

### CROSS-REFERENCE TO RELATED APPLICATIONS

This application is a continuation of International Application No. PCT/US2024/041133, filed Aug. 6, 2024, which claims benefit to U.S. Provisional Application No. 63/530,980, filed Aug. 6, 2023, each of which is incorporated herein by reference in its entirety.

### TECHNICAL FIELD

The present disclosure, in at least some embodiments, relates to methods and systems for gathering physiometric data, and more specifically to methods and systems for gathering user physiometric data from a dental scan.

### BACKGROUND

Dental professionals may treat and monitor a patient's dental condition based on in-person visits. Treatment and monitoring of a patient's dental condition may require preforming a dental imagining to provide the dentist penetration look of the teeth and jaw's condition. During the in-person visits, the dental professional can be exposed to the patient health condition. The quality of treatment and the accuracy of monitoring may vary depending on availability of such dental imaging. In some cases, suboptimal treatment outcomes may result if a patient is unable or unwilling to schedule regular visits that can provide dental imaging.

### SUMMARY

Recognized herein is a need for dental monitoring solutions to allow dental assessment, optionally remote dental assessment, without requiring a dental professional to be physically present with the patient. However, such conventional teledentistry solutions may provide inadequate levels of supervision. Further, such conventional teledentistry solutions may be limited by an inaccurate or insufficient monitoring of a patient's dental condition based on one or more photos taken by the patient, if the photos do not adequately capture various intraoral features.

The present disclosure provides methods and systems for dental assessment, such as remote dental assessment. As used herein, the term “remote dental assessment” may refer to assessments conducted by remote personnel and may refer to the acquisition of one or more intraoral videos and/or intraoral images that can be assessed using a remote computing device, or can be sent to different remote locations for assessment. The methods and systems disclosed herein may provide a convenient solution to assess oral treatment progression and user experience for dental assessment presentation, in some embodiments, the methods and system disclosed herein may provide a solution to gather user physiometric data from a dental scan, real time or post time.

In an aspect, provided herein is a method of gathering physiometric data of a subject, comprising: (a) providing a dental scan of an intraoral region of the subject; (b) analyzing one or more properties of the dental scan; and (c) generating the physiometric data of the subject based on the one or more properties of the dental scan.

In some cases, the dental scan is acquired using a mobile digital camera and a hollow tubular adapter, wherein a first opening of the hollow tubular adapter is attached to the mobile digital camera, and wherein a second opening of the hollow tubular adapter is configured to contact lips of the subject. In some cases, the mobile digital camera comprises a light source configured to emit light in the visible spectrum.

In some cases, the mobile digital camera comprises a light source configured to emit light in the ultraviolet (UV) spectrum. In some cases, the mobile digital camera comprises a light source configured to emit light in the infrared (IR) spectrum. In some cases, the adapter comprises a light source configured to emit light in the visible spectrum. In some cases, the adapter comprises a light source configured to emit light in the ultraviolet (UV) spectrum. In some cases, the adapter comprises a light source configured to emit light in the infrared (IR) spectrum.

In some cases, the adapter comprises a calibration target. In some cases, the calibration target is configured to align the dental scan with an intraoral landmark of the subject.

In some cases, the physiometric data comprises body temperature, blood pressure, pulse, breathing rate, breathing volume, blood flow, hydration, oxygen saturation, blood sugar, or blood cell volume of the subject, or any combination thereof. In some cases, the one or more properties comprise a color of one or more tissues in the intraoral region of the subject. In some cases, the one or more properties comprise an absorbance spectra of one or more tissues in the intraoral region of the subject. In some cases, the one or more properties comprise a spectral distribution of one or more tissues in the intraoral region of the subject. In some cases, wherein the one or more properties comprise a size of one or more tissues in the intraoral region of the subject. In some cases, the method further comprises calibration with the calibration target. In some cases, the one or more properties comprise an appearance of one or more tissues in the intraoral region of the subject. In some cases, the one or more properties comprise a shape of one or more tissues in the intraoral region of the subject. In some cases, the one or more properties comprise a height of one or more tissues in the intraoral region of the subject. In some cases, the one or more properties comprise a dent or a dentition of one or more tissues in the intraoral region of the subject.

In some cases, the analyzing further comprises adaptation of one or more tissues in the intraoral region of the subject. In some cases, the adaptation comprises projection of the tissue location on a 3D Model. In some cases, the intraoral region of the subject comprises soft tissue or hard tissue. In some cases, the dental scan comprises one or more videos or images. In some cases, the one or more properties are of a tissue that is located in at least two images or at least two video frames.

In some cases, the generating comprises calculating a difference between at least two video frames or at least two images. In some cases, the generating comprises performing a statistical analysis of the differences between the at least two video frames or the at least two images. In some cases, the physiometric data is generated during performance of the dental scan. In some cases, the dental scan is provided from a data set. In some cases, the generating further comprises providing additional physiometric data of the subject. In some cases, the generating comprises comparing the dental scan to a previous dental scan of the subject.

In some cases, the hollow tubular adapter comprises a photoplethysmogram (PPG) sensor. In some cases, the PPG sensor is configured to contact a soft tissue of the intraoral region of the subject. In some cases, the hollow tubular adapter comprises one or more electrodes.

In another aspect, provided herein is a system for gathering physiometric data of a subject, comprising: a processor; a storage element connected to the processor; encoded instructions stored in the storage element, wherein the encoded instructions when implemented by the processor, configure the system to: (a) provide a dental scan of an intraoral region of the subject; (b) analyze one or more properties of the dental scan; and (c) generate the physiometric data of the subject based on the one or more properties of the dental scan.

In some cases, the dental scan is acquired using a mobile digital camera and a hollow tubular adapter, wherein a first opening of the hollow tubular adapter is attached to the mobile digital camera, and wherein a second opening of the hollow tubular adapter is configured to contact lips of the subject.

In some cases, the mobile digital camera comprises a light source configured to emit light in the visible spectrum. In some cases, the mobile digital camera comprises a light source configured to emit light in the ultraviolet (UV) spectrum. In some cases, the mobile digital camera comprises a light source configured to emit light in the infrared (IR) spectrum. In some cases, the adapter comprises a light source configured to emit light in the visible spectrum. In some cases, the adapter comprises a light source configured to emit light in the ultraviolet (UV) spectrum. In some cases, the adapter comprises a light source configured to emit light in the infrared (IR) spectrum. In some cases, the adapter comprises a calibration target. In some cases, the calibration target is configured to align the dental scan with an intraoral landmark of the subject.

In some cases, the physiometric data comprises body temperature, blood pressure, pulse, breathing rate, breathing volume, blood flow, hydration, oxygen saturation, blood sugar, or blood cell volume of the subject, or any combination thereof. In some cases, the one or more properties comprise a color of one or more tissues in the intraoral region of the subject. In some cases, the one or more properties comprise an absorbance spectra of one or more tissues in the intraoral region of the subject. In some cases, the one or more properties comprise a spectral distribution of one or more tissues in the intraoral region of the subject. In some cases, the one or more properties comprise a size of one or more tissues in the intraoral region of the subject. In some cases, the method further comprises calibration with the calibration target. In some cases, the one or more properties comprise an appearance of one or more tissues in the intraoral region of the subject. In some cases, the one or more properties comprise a shape of one or more tissues in the intraoral region of the subject. In some cases, one or more properties comprise a height of one or more tissues in the intraoral region of the subject. In some cases, the one or more properties comprise a dent or a dentition of one or more tissues in the intraoral region of the subject.

In some cases, the analyzing further comprises adaptation of one or more tissues in the intraoral region of the subject. In some cases, the adaptation comprises projection of the tissue location on a 3D Model. In some cases, the intraoral region of the subject comprises soft tissue or hard tissue. In some cases, the dental scan comprises one or more videos or images. In some cases, the one or more properties are of a tissue that is located in at least two images or at least two video frames. In some cases, the generating comprises calculating a differences between at least two video frames or at least two images. In some cases, the generating comprises performing a statistical analysis of the differences between the at least two video frames or the at least two images. In some cases, the physiometric data is generated during performance of the dental scan. In some cases, the dental scan is provided from a data set. In some cases, the generating further comprises providing additional physiometric data of the subject. In some cases, the generating comprises comparing the dental scan to a previous dental scan of the subject.

In some cases, the hollow tubular adapter comprises a photoplethysmogram (PPG) sensor. In some cases, the PPG sensor is configured to contact a soft tissue of the intraoral region of the subject. In some cases, the hollow tubular adapter comprises one or more electrodes.

In another aspect, provided herein is a hollow tubular adapter, comprising: a photoplethysmogram (PPG) sensor; at least two electrodes; and a connection module, wherein a first opening of the hollow tubular adapter is configured to attach to a mobile digital device, and wherein a second opening of the hollow tubular adapter is configured to contact lips of the subject.

In some cases, the photoplethysmogram (PPG) sensor is configured to face an intraoral soft tissue of the subject. In some cases, at least one electrode of the at least two electrodes is configured to face an intraoral soft tissue of the subject. In some cases, the photoplethysmogram (PPG) sensor and the at least two electrodes are connected to the connection module. In some cases, the connection module comprises Bluetooth or Wi-Fi connectivity. In some cases, the mobile digital device is a smartphone. In some cases, the connection module is configured to transfer data from the hollow tubular adapter to the smartphone.

Unless otherwise defined, all technical and scientific terms used herein have the same meaning as commonly understood by one of ordinary skill in the art to which this invention belongs. Although suitable methods and materials are described below, methods and materials similar or equivalent to those described herein can be used in the practice of the present invention. In case of conflict, the patent specification, including definitions, will control. All materials, methods, and examples are illustrative only and are not intended to be limiting.

As used herein, the terms “comprising” and “including” or grammatical variants thereof are to be taken as specifying inclusion of the stated features, integers, actions or components without precluding the addition of one or more additional features, integers, actions, components or groups thereof. This term is broader than, and includes the terms “consisting of” and “consisting essentially of” as defined by the Manual of Patent Examination Procedure of the United States Patent and Trademark Office.

The term “method” refers to manners, means, techniques and procedures for accomplishing a given task including, but not limited to, those manners, means, techniques and procedures either known to, or readily developed from known manners, means, techniques and procedures by practitioners of architecture and/or computer science.

Implementation of the method and system of the present invention involves performing or completing selected tasks or steps manually, automatically, or a combination thereof. Moreover, according to actual instrumentation and equipment of preferred embodiments of methods, apparatus and systems of the present invention, several selected steps could be implemented by hardware or by software on any operating system of any firmware or a combination thereof. For example, as hardware, selected steps of the invention could be implemented as a chip or a circuit. As software, selected steps of the invention could be implemented as a plurality of software instructions being executed by a computer using any suitable operating system. In any case, selected steps of the method and system of the invention could be described as being performed by a data processor, such as a computing platform for executing a plurality of instructions.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF THE DRAWINGS

In order to understand the invention and to see how it may be carried out in practice, embodiments will now be described, by way of non-limiting examples only, with reference to the accompanying figures. In the figures, identical and similar structures, elements or parts thereof that appear in more than one figure are generally labeled with the same or similar references in the figures in which they appear. Dimensions of components and features shown in the figures are chosen primarily for convenience and clarity of presentation and are not necessarily to scale. The attached figures are:

FIG. 1 is a flow-chart schematically illustrating methods for gathering user physiometric data from a dental scan, in accordance with some embodiments.

FIG. 2 schematically illustrates example steps in gathering user physiometric data from soft tissues from a dental scan, in accordance with some embodiments.

FIG. 3 schematically illustrates example steps in gathering user physiometric data from hard tissues from a dental scan, in accordance with some embodiments.

FIG. 4 schematically illustrates a computer system that is programmed or otherwise configured to implement at least some of the methods disclosed herein, in accordance some embodiments.

FIGS. 5A and 5B schematically illustrate an example of a hollow tubular adapter comprising a sensor, in accordance with some embodiments.

### DETAILED DESCRIPTION

While various embodiments of the invention have been shown and described herein, it will be obvious to those skilled in the art that such embodiments are provided by way of example only. Numerous variations, changes, and substitutions may occur to those skilled in the art without departing from the invention. It should be understood that various alternatives to the embodiments of the invention described herein may be employed.

The term “real-time,” as used herein, generally refers to a simultaneous or substantially simultaneous occurrence of a first event or action with respect to an occurrence of a second event or action. A real-time action or event may be performed within a response time of less than one or more of the following: ten seconds, five seconds, one second, a tenth of a second, a hundredth of a second, a millisecond, or less relative to at least another event or action. A real-time action may be performed by one or more computer processors.

As used herein, when a numerical value is preceded by the term “about”, the term “about” is intended to indicate +/−10% of that value. Whenever the term “at least,” “greater than,” or “greater than or equal to” precedes the first numerical value in a series of two or more numerical values, the term “at least,” “greater than” or “greater than or equal to” applies to each of the numerical values in that series of numerical values. For example, greater than or equal to 1, 2, or 3 is equivalent to greater than or equal to 1, greater than or equal to 2, or greater than or equal to 3.

Whenever the term “no more than,” “less than,” or “less than or equal to” precedes the first numerical value in a series of two or more numerical values, the term “no more than,” “less than,” or “less than or equal to” applies to each of the numerical values in that series of numerical values. For example, less than or equal to 3, 2, or 1 is equivalent to less than or equal to 3, less than or equal to 2, or less than or equal to 1.

Whenever the term “more than,” “at least,” or “more than or equal to” precedes the first numerical value in a series of two or more numerical values, for example, more than or at least one equals to 1, 2, 10 or even more and it can represent even hundred or more.

The terms “a,” “an,” and “the,” as used herein, generally refer to singular and plural references unless the context clearly dictates otherwise.

### Overview

According to an aspect, the present disclosure provides methods and systems for gathering user physiometric data from a dental scan. As used herein the term “physiometric data” generally refers to any life sign that can be measured from a subject. “Physiometric data” can include the elementary “vital signs” such as body temperature, heart pulse, respiration rate, and blood pressure. “Physiometric data” can also include measurements such as blood oxygen saturation, blood glucose, breathing volume, blood flow, hydration, and blood cell volume. The methods and systems disclosed herein may provide a convenient and simple way to gather user physiometric data from a dental scan or during a dental scan. The methods and systems disclosed herein may provide dentists and orthodontists or any health care personal with an analysis of the patient's condition based on a video or on one or more images of a dental scan. In some embodiments, the dental scan is captured remotely by a user. In some embodiments, the user is the subject of the dental scan. In some embodiments, the user is a person in a location remote from the dental provider.

As used herein the term “dental scope” or “dental adapter” generally refers to a device that can be attached to a camera or mobile phone comprising a camera and enables capture of at least one intraoral image or video of a subject. The term “dental scope” or “dental adapter” may refer to a hollow tubular adapter. The hollow tubular adapter can have a first opening that is configured to attach to the mobile digital camera. The hollow tubular adapter can have a second opening that is configured to contact the lips of a user. An example of a hollow tubular adapter that can be used with the systems and methods described herein is described in U.S. patent application Ser. No. 17/336,997 and PCT Application No. WO2023009763.

As used herein, the terms “dental scan” or “oral scan” generally refers to intraoral videos or intraoral images, or oral video, or oral images that are the results of using the “dental scope” or “dental adapter” as attached to a digital camera such as mobile device while acquiring an intraoral video or intraoral image. An example dental scan, and possible uses, can be seen in U.S. patent application Ser. No. 17/336,997. The intraoral videos or intraoral images or the dental scan provided to the methods and systems of the present disclosure may be implemented using a software application that is configured to enable a dental patient to capture videos and/or images of intraoral regions. The software application may be used by a user or a subject (e.g., a dental patient) in conjunction with a mobile device to remotely monitor a intraoral condition of the subject.

As used herein, “remote monitoring” may generally refer to monitoring the intraoral region of a subject and/or a condition of an intraoral region of a subject, wherein the monitoring is performed at one or more locations remote from the subject. For example, a dentist or a medical specialist may monitor the intraoral anatomy or intraoral condition at a first location that is different than a second location at which the subject is located. The first location and the second location may be separated by a distance spanning at least 1 meter, 1 kilometer, 10 kilometers, 100 kilometers, 1000 kilometers, or more.

The remote monitoring may be performed by assessing an intraoral condition or physiometric data of the subject using one or more dental scans captured from the subject when the subject is located remotely from the dentist or a dental office. In some cases, the remote monitoring may be performed in real-time such that a dentist is able to assess the dental condition or physiometric data of a subject when a subject uses a mobile device to acquire one or more videos or images of one or more intraoral regions in the subject's mouth. The remote monitoring may be performed using equipment, hardware, and/or software that is not physically located at a dental office.

Software applications for dental assessment may be configured to run on a mobile device. The mobile device may comprise a smartphone, a tablet, a laptop, or any suitable device that may be used by a patient to capture one or more dental assessments. The software application may be installed on a mobile device of a user. The software application may be a patient-side software application. Alternatively, the software application for dental assessment may be configured to run on a fixed-location device, such as a desktop computer. Alternatively, the software application for dental assessment may be configured to run on a remote location device, such as a compute cloud server or service.

The methods and systems disclosed herein may provide a convenient solution to assess oral treatment progression and user experience for the collection and presentation of user physiometric data. In some embodiments, the methods and systems disclosed herein may provide a solution for comparing oral representation of a subject at different times to represent oral progression of the subject.

Methods for Gathering User Physiometric Data from a Dental Scan

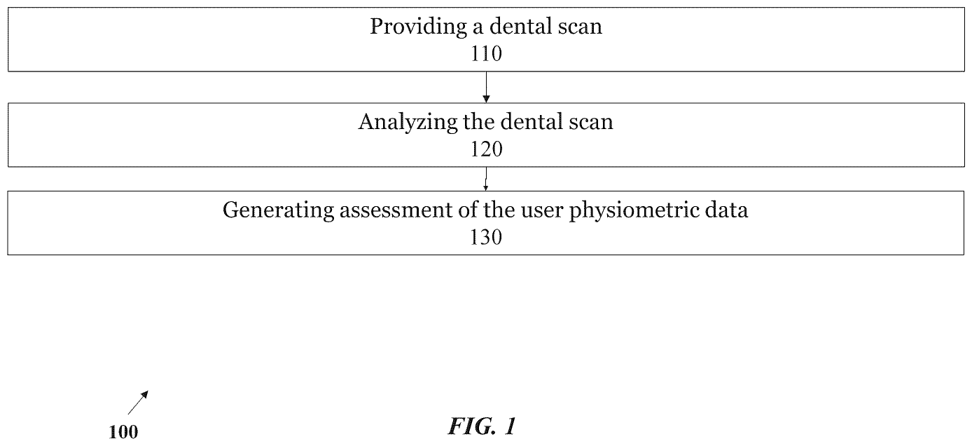

FIG. 1 schematically illustrates methods 100 to gather user physiometric data from a dental scan, in accordance with some embodiments. In some cases, the method includes providing a dental scan 110; analyzing the dental scan 120; and generating assessment on the user physiometric data 130.

According to some embodiments, the dental scan 110 was acquired using a mobile digital camera and a hollow tubular adapter. The hollow tubular adapter can have a first opening that is configured to attach to the mobile digital camera. The hollow tubular adapter can have a second opening that is configured to contact the lips of a user. An example of a hollow tubular adapter that can be used with the systems and methods described herein is described in U.S. patent application Ser. No. 17/336,997 and PCT Application No. WO2023009763.

According to some embodiments, the mobile digital camera comprises a light source in the visible spectrum.

According to some embodiments, the mobile digital camera comprises a light source in the ultraviolet (UV) spectrum.

According to some embodiments, the mobile digital camera comprises a light source in the infrared (IR) spectrum.

According to some embodiments, the adapter comprises a light source in the visible spectrum.

According to some embodiments, the adapter comprises a light source in the ultraviolet (UV) spectrum.

According to some embodiments, the adapter comprises light source in the infrared (IR) spectrum.

According to some embodiments, the adapter comprises a calibration target. According to some embodiments, the calibration target comprises at least one reference color. According to some embodiments, the calibration target comprises at least one reference pattern. According to some embodiments, the calibration target comprises at least one preset temperature reference.

According to some embodiments, the dental scan 110 comprises a video or one or more images. According to some embodiments, at least one location from the intraoral region appears on two or more images. According to some embodiments, the dental scan video can be separated into at least two video frames or images.

According to some embodiments, the physiometric data includes at least one of: body temperature, blood pressure, pulse, breathing rate, breathing volume, blood flow, hydration, oxygen saturation, blood sugar, and blood cell volume.

According to some embodiments, the analyzing 120 comprises analyzing differences in color of tissue at a location that appears in at least two video frames or at least two images. According to some embodiments, the differences in color are evaluated at the location. In some embodiments, the differences in color are evaluated at an area surrounding the location.

According to some embodiments the analyzing 120 comprises analyzing differences in absorbance of tissue at a location that appears in at least two video frames or at least two images. According to some embodiments, the differences in absorbance are evaluated at the location. In some embodiments, the differences in absorbance are evaluated at an area surrounding the location.

According to some embodiments, the analyzing 120 comprises analyzing differences in spectral distribution of tissue at a location that appears in at least two video frames or at least two images. According to some embodiments, the differences in light spectral distribution are evaluated at the location. In some embodiments, the differences in light spectral distribution are evaluated at an area surrounding the location.

According to some embodiments, the analyzing 120 comprises analyzing differences in size of tissue at a location that appears in at least two video frames or at least two images. According to some embodiments, the differences in the size of tissue are evaluated at the location. In some embodiments, the differences in the size of tissue are evaluated at an area surrounding the location.

According to some embodiments, the analyzing 120 comprises calibration with the calibration target.

According to some embodiments, the analyzing 120 comprises analyzing differences in appearance of tissue at a location that appears in at least two video frames or at least two images. According to some embodiments, the differences in appearance are evaluated at the location. In some embodiments, the differences in appearance are evaluated at an area surrounding the location.

According to some embodiments, the analyzing 120 comprises analyzing differences in shape of tissue at a location that appears in at least two video frames or at least two images. According to some embodiments, the differences in shape are evaluated at the location. In some embodiments, the differences in shape are evaluated at an area surrounding the location.

According to some embodiments, the analyzing 120 comprises analyzing differences in height of tissue at a location that appears in at least two video frames or at least two images. According to some embodiments, the differences in height of the tissue are evaluated at the location. In some embodiments, the differences in height of the tissue are evaluated at an area surrounding the location.

According to some embodiments, the analyzing 120 comprises analyzing differences dent/dentition of tissue at a location that appears in at least two video frames or at least two images. According to some embodiments, the differences in the dent/dentition are evaluated at the location. In some embodiments, the differences in dent/dentition are evaluated at an area surrounding the location.

According to some embodiments, the analyzing 120 comprises adaptation of the tissue location in at least two video frames or at least two images.

According to some embodiments, the adaptation comprises projection of the tissue location on a 3D Model.

According to some embodiments, the analyzing 120 comprises analyzing soft tissue or hard tissue.

According to some embodiments, the generation 130 comprises calculation of differences in at least two video frames or at least two images.

According to some embodiments, the generation 130 comprises calculation of differences between at least two sets of at least two video frames or at least two images.

According to some embodiments, the generation 130 comprises a statistical analysis of differences of more than two video frames or more than two images.

According to some embodiments, the physiometric data is generated during a dental scan.

According to some embodiments the dental scan is provided from a data set.

According to some embodiments, the generation 130 comprises providing additional physiometric data input.

According to some embodiments, the generation 130 comprises comparing to previous user's dental scan and/or physiometric data.

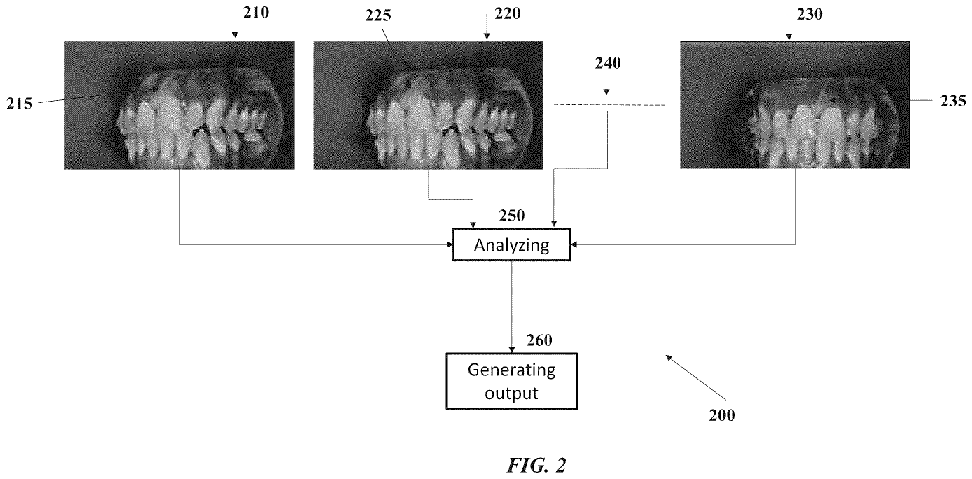

FIG. 2 schematically illustrates 200 example steps in gathering user physiometric data from soft tissues of a dental scan, in accordance some embodiments. The steps can include receiving a user dental scan, which can comprise a video or images. The user dental scan can be represented in at least two of images 210, 220, and 230. The images 210, 220, and 230 can be frames of the dental scan video, or a series of images of a dental scan. As shown in FIG. 2, the image 220 can be the next frame after frame 210 in a dental scan video. The dotted line 240 can represent all of the frames from the frame 220 until the last frame of this dental scan video, image 230. Locations 215, 225, 235 on images 210, 220, 230 point to a specific location in the user's intraoral area. In FIG. 2, the example selected location 215, 225, 235 is a soft tissue in the front upper gingiva just next to the root side border of tooth 21. Any other location of soft tissue that appears on all the images can be selected for this example.

At FIG. 2 for example, locations 215, 225 and/or 235 or the surrounding locations can undergo analyzing 250. The analyzing may take into account at least one of the appearance differences, color differences, absorbance differences, spectral distribution, size differences, shape differences, height differences or dent/dentition differences. Analyzing can be performed with or without referring to a calibration target. The one or more differences from at least two images 210, 220, and 230 can be used to measure blood flow using transdermal imaging.

At FIG. 2 example, locations 215, 225 and/or 235 or the surrounding locations can undergo analyzing 250. The analyzing may take into account at least one of the appearance differences, color differences, absorbance differences, or spectral distribution, or any combination thereof. Analyzing can be performed with or without referring to a calibration target. The one or more differences from at least two images 210, 220, and 230 can be used to assess oxygenation level, and body heat using NIR or UV filters.

The generated output 260 can be a direct result of analyzing 250. In some cases, analyzing 250 takes into account external data. The external data can be a known or measured physiometric measurements of the user. Analyzing 250 results can lead to determination of a user's body temperature, blood pressure, pulse, breathing rate, breathing volume, blood flow, hydration, oxygen saturation, blood sugar, and blood cell volume.

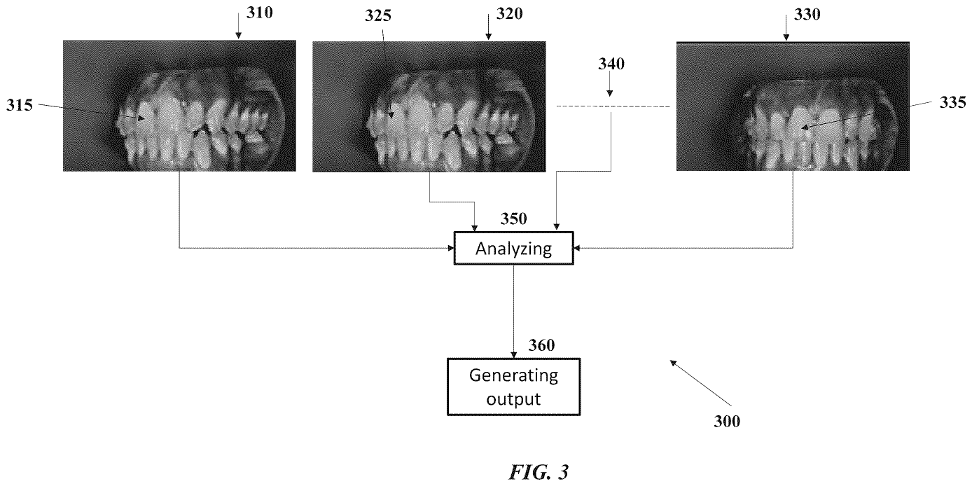

FIG. 3 schematically illustrates 300 example steps in gathering user physiometric data from hard tissue of a dental scan. The steps can include receiving a user dental scan which can comprise a video or images. The user dental scan can be represented in at least two of images 310, 320, and 330. The images 310, 320, and 330 can be frames of the dental scan video, or a series of images of dental scan. As shown in FIG. 3, the image 320 can be the next frame after frame 310 in a dental scan video. The dotted line 340 can represent all of the frames from the frame 320 until the last frame of this dental scan video, image 330. Locations 315, 325, 335 on images 310, 320, 330 point to a specific location in the user's intraoral area. In FIG. 3, the example selected location 315, 325, and 335 is a hard tissue tooth 11 in the center, at the front upper arch. Any other location of hard tissue that appears on all the images can be selected for this example.

At FIG. 3 for example, locations 315, 325 and/or 335 or the surrounding locations can undergo analyzing 350. The analyzing may take into account at least one of the appearance differences, color differences, absorbance differences, spectral distribution with or without referring to calibration target, those one or more differences from at least two images 310, 320 and 330 can be used to measure breathing rate, breathing volume, or hydration using transdermal imaging.

At FIG. 3 example, locations 315, 325 and/or 335 or the surrounding location can undergo analyzing. The analyzing may take into account at least one of the appearance differences, color differences, absorbance differences, or spectral distribution, or any combination thereof. Analyzing can be performed with or without referring to a calibration target. The one or more differences from at least two images 310, 320, and 330 can be used to assess breathing rate, breathing volume, or hydration using NIR or UV filters.

The generated output 360 can be a direct result of analyzing 350. In some cases, analyzing 350 takes into account external data. The external data can be a known or measured physiometric measurements of the user. Analyzing 350 results can lead to determination of a user's of body temperature, breathing rate, breathing volume, hydration and more.

System to Gather User Physiometric Data from a Dental Scan.

According to an aspect described herein, there is provided a computer implemented system to gather user physiometric data from a dental scan, comprising: providing a dental scan; analyzing the dental scan; and generating an assessment of the user physiometric data.

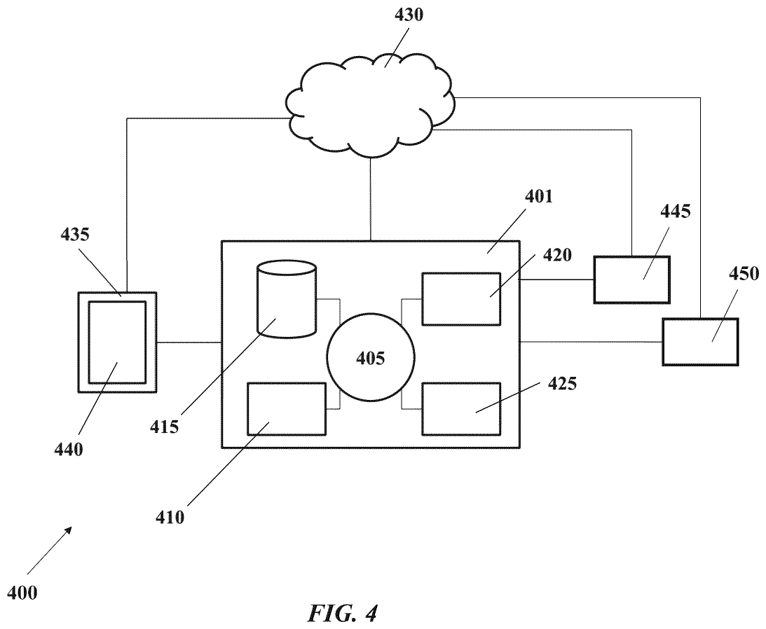

FIG. 4 shows a computer system 401 that is programmed or otherwise configured to gather user physiometric data from a dental scan. The computer system 401 may be configured to, for example, process intraoral videos or images captured using the camera of a mobile device, and presenting dental scans, intraoral models or oral representations. The computer system 401 can be an electronic device of a user or a computer system that is remotely located with respect to the electronic device, or even in the cloud. The electronic device can be a mobile electronic device. The computer system 401 can be a smartphone.

The computer system 401 may include a central processing unit (CPU, also “processor” and “computer processor” herein) 405, which can be a single core or multi core processor, or a plurality of processors for parallel processing. The computer system 401 also includes memory or memory location 410 (e.g., random-access memory, read-only memory, flash memory), electronic storage unit 415 (e.g., hard disk, Solid State drive or equivalent storge unit), communication interface 420 (e.g., network adaptor) for communicating with one or more other systems, and peripheral devices 425, such as cache, other memory, data storage and/or electronic display adaptors. The memory 410, storage unit 415, interface 420 and peripheral devices 425 are in communication with the CPU 405 through a communication bus (solid lines), such as a motherboard. The storage unit 415 can be a data storage unit (or data repository) for storing data. The computer system 401 can be operatively coupled to a computer network (“network”) 430 with the aid of the communication interface 420. The network 430 can be the Internet, an internet and/or extranet, or an intranet and/or extranet that is in communication with the Internet. The network 430 in some cases is a telecommunication and/or data network. The network 430 can include one or more computer servers, which can enable distributed computing, such as cloud computing. The network 430, in some cases with the aid of the computer system 401, can implement a peer-to-peer network, which may enable devices coupled to the computer system 401 to behave as a client or a server.

The CPU 405 can execute a sequence of machine-readable instructions, which can be embodied in a program or software. The instructions may be stored in a memory location, such as the memory 410. The instructions can be directed to the CPU 405, which can subsequently program or otherwise configure the CPU 405 to implement methods of the present disclosure. Examples of operations performed by the CPU 405 can include fetch, decode, execute, and writeback.

The CPU 405 can be part of a circuit, such as an integrated circuit. One or more other components of the system 401 can be included in the circuit. In some cases, the circuit is an application specific integrated circuit (ASIC).

The storage unit 415 can store files, such as drivers, libraries and saved programs. The storage unit 415 can store user data, e.g., user preferences and user programs. The computer system 401 in some cases can include one or more additional data storage units that are located external to the computer system 401 (e.g., on a remote server that is in communication with the computer system 401 through an intranet or the Internet).

The computer system 401 can communicate with one or more remote computer systems through the network 430. For instance, the computer system 401 can communicate with a remote computer system of a user (e.g., a subject, a dental patient, or a dentist). Examples of remote computer systems include personal computers (e.g., portable PC), slate or tablet PC's (e.g., Apple® iPad, Samsung® Galaxy Tab), telephones, Smart phones (e.g., Apple® iPhone, Android-enabled device, Blackberry®), or personal digital assistants. The user can access the computer system 401 via the network 430.

Methods and systems as described herein can be implemented by way of machine (e.g., computer processor) executable code stored on an electronic storage location of the computer system 401, such as, for example, on the memory 410 or electronic storage unit 415. The machine executable or machine-readable code can be provided in the form of software. During use, the code can be executed by the processor 405. In some cases, the code can be retrieved from the storage unit 415 and stored on the memory 410 for ready access by the processor 405. In some situations, the electronic storage unit 415 can be precluded, and machine-executable instructions are stored on memory 410.

The code can be pre-compiled and configured for use with a machine having a processor adapted to execute the code, or can be compiled during runtime. The code can be supplied in a programming language that can be selected to enable the code to execute in a pre-compiled or as-compiled fashion.

Aspects of the systems and methods provided herein, such as the computer system 401, can be embodied in programming. Various aspects of the technology may be thought of as “products” or “articles of manufacture” typically in the form of machine (or processor) executable code and/or associated data that is carried on or embodied in a type of machine-readable medium. Machine-executable code can be stored on an electronic storage unit, such as memory (e.g., read-only memory, random-access memory, flash memory) or a storage unit. “Storage” type media can include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical and electromagnetic waves, such as used across physical interfaces between local devices, through wired and optical landline networks and over various air-links. The physical elements that carry such waves, such as wired or wireless links, optical links or the like, also may be considered as media bearing the software. As used herein, unless restricted to non-transitory, tangible “storage” media, terms such as computer or machine “readable medium” refer to any medium that participates in providing instructions to a processor for execution.

Hence, a machine-readable medium, such as computer-executable code, may take many forms, including but not limited to, a tangible storage medium, a carrier wave medium or physical transmission medium. Non-volatile storage media including, for example, optical or magnetic disks, or any storage devices in any computer(s) or the like, may be used to implement the databases, etc. shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

The computer system 401 can include or be in communication with an electronic display 435 that comprises a user interface (UI) 440 for providing, for example, a portal for a subject or a user to view one or more intraoral images or videos captured using a mobile device of the subject or the user. In some cases, the electronic display 435 may be the feedback element providing the generated output, for example displaying message or shape or light in accordance to some embodiments. The portal may be provided through an application programming interface (API). A user or entity can also interact with various elements in the portal via the UI. Examples of UI's include, without limitation, a graphical user interface (GUI) and web-based user interface.

The computer system 401 can include or be in communication with a camera 445 for providing, for example, ability to capture videos or images of the intraoral of a user. Methods and systems of the present disclosure can be implemented by way of one or more algorithms. An algorithm can be implemented by way of software upon execution by the central processing unit 405. The algorithm can, for example, implement a method for presenting teeth morphology. The method may comprise processing videos or images captured using the camera of the mobile device or processing received user oral video or oral images and/or received user dental imaging image and executed to generate output.

The algorithm can, for example, implement a method of machine learning framework to generate data between two oral representations. In order to transfer a given capability into a machine, so-called supervised machine-learning techniques can be employed. Broadly speaking, a training dataset containing input images together with requested output (the “ground-truth”) is assembled. This dataset is assembled from input images which should resemble the expected input images to the machine after deployment as much as possible. The requested output can be any desired output type, e.g image classification, segmentation mask, image augmentation, text etc. The requested output can be gathered by manual human labeling, or by fusing data with other means of data gathering. This dataset is then fed into a machine-learning framework which uses an optimization process to control internal parameters in a model architecture (e.g SVM, neural network, random forest etc.). The optimization process goal is to reduce the error between the machine's output and the ground-truth output for each image in the training dataset. Once the optimization process has reached the required accuracy on the training dataset the machine's performance can be tested on a similar test dataset to validate the performance in real-world settings.

According to some embodiments, there is provided a system 401 to gather user physiometric data from a dental scan, comprising: a processor 405; a storage element 415 connected to the processor 405; encoded instructions stored in the storage element 415, a digital display 435 and an ability to receive user input 440, wherein the encoded instructions when implemented by the processor 405, configure the system 401 to: receive or provide a dental scan of a user; analyzing the dental scan; and, generating assessment on the user physiometric data.

According to some embodiments, the dental scan 110 was acquired using a mobile digital camera and a hollow tubular adapter. The hollow tubular adapter can have a first opening that is configured to attach to the mobile digital camera. The hollow tubular adapter can have a second opening that is configured to contact the lips of a user. An example of a hollow tubular adapter that can be used with the systems and methods described herein is described in U.S. patent application Ser. No. 17/336,997 and PCT Application No. WO2023009763.

According to some embodiments, the mobile digital camera comprises a light source in the visible spectrum.

According to some embodiments, the mobile digital camera comprises a light source in the ultraviolet (UV) spectrum.

According to some embodiments, the mobile digital camera comprises a light source in the infrared (IR) spectrum.

According to some embodiments, the adapter comprises a light source in the visible spectrum.

According to some embodiments, the adapter comprises a light source in the ultraviolet (UV) spectrum.

According to some embodiments, the adapter comprises light source in the infrared (IR) spectrum.

According to some embodiments, the adapter comprises a calibration target. According to some embodiments, the calibration target comprises at least one reference color. According to some embodiments, the calibration target comprises at least one reference pattern. According to some embodiments, the calibration target comprises at least one preset temperature reference.

According to some embodiments, the dental scan 110 comprises a video or one or more images. According to some embodiments, at least one location from the intraoral region appears on two or more images. According to some embodiments, the dental scan video can be separated into at least two video frames or images.

According to some embodiments, the physiometric data includes at least one of: body temperature, blood pressure, pulse, breathing rate, breathing volume, blood flow, hydration, oxygen saturation, blood sugar, and blood cell volume.

According to some embodiments, the analyzing 120 comprises analyzing differences in color of tissue at a location that appears in at least two video frames or at least two images. According to some embodiments, the differences in color are evaluated at the location. In some embodiments, the differences in color are evaluated at an area surrounding the location.

According to some embodiments the analyzing 120 comprises analyzing differences in absorbance of tissue at a location that appears in at least two video frames or at least two images. According to some embodiments, the differences in absorbance are evaluated at the location. In some embodiments, the differences in absorbance are evaluated at an area surrounding the location.

According to some embodiments, the analyzing 120 comprises analyzing differences in spectral distribution of tissue at a location that appears in at least two video frames or at least two images. According to some embodiments, the differences in light spectral distribution are evaluated at the location. In some embodiments, the differences in light spectral distribution are evaluated at an area surrounding the location.

According to some embodiments, the analyzing 120 comprises analyzing differences in size of tissue at a location that appears in at least two video frames or at least two images. According to some embodiments, the differences in the size of tissue are evaluated at the location. In some embodiments, the differences in the size of tissue are evaluated at an area surrounding the location.

According to some embodiments, the analyzing 120 comprises calibration with the calibration target.

According to some embodiments, the analyzing 120 comprises analyzing differences in appearance of tissue at a location that appears in at least two video frames or at least two images. According to some embodiments, the differences in appearance are evaluated at the location. In some embodiments, the differences in appearance are evaluated at an area surrounding the location.

According to some embodiments, the analyzing 120 comprises analyzing differences in shape of tissue at a location that appears in at least two video frames or at least two images. According to some embodiments, the differences in shape are evaluated at the location. In some embodiments, the differences in shape are evaluated at an area surrounding the location.

According to some embodiments, the analyzing 120 comprises analyzing differences in height of tissue at a location that appears in at least two video frames or at least two images. According to some embodiments, the differences in height of the tissue are evaluated at the location. In some embodiments, the differences in height of the tissue are evaluated at an area surrounding the location.

According to some embodiments, the analyzing 120 comprises analyzing differences dent/dentition of tissue at a location that appears in at least two video frames or at least two images. According to some embodiments, the differences in the dent/dentition are evaluated at the location. In some embodiments, the differences in dent/dentition are evaluated at an area surrounding the location.

According to some embodiments, the analyzing 120 comprises adaptation of the tissue location in at least two video frames or at least two images.

According to some embodiments, the adaptation comprises projection of the tissue location on a 3D Model.

According to some embodiments, the analyzing 120 comprises analyzing soft tissue or hard tissue.

According to some embodiments, the generation 130 comprises calculation of differences in at least two video frames or at least two images.

According to some embodiments, the generation 130 comprises calculation of differences between at least two sets of at least two video frames or at least two images.

According to some embodiments, the generation 130 comprises a statistical analysis of differences of more than two video frames or more than two images.

According to some embodiments, the physiometric data is generated during a dental scan.

According to some embodiments the dental scan is provided from a data set.

According to some embodiments, the generation 130 comprises providing additional physiometric data input.

According to some embodiments, the generation 130 comprises comparing to previous user's dental scan and/or physiometric data.

### Hollow Tubular Adapter to Gather Physiometric Data

According to an embodiment described herein, there is provided a hollow tubular adapter, comprising: a photoplethysmogram (PPG) sensor; at least two electrodes; and a connection module. In some cases, one opening of the hollow tubular adapter can be attached to a mobile digital device, and the second opening of the hollow tubular adapter can be in contact with the user's inner lips. In some cases, the lips lay on the outer of the second opening of the hollow tubular adapter.

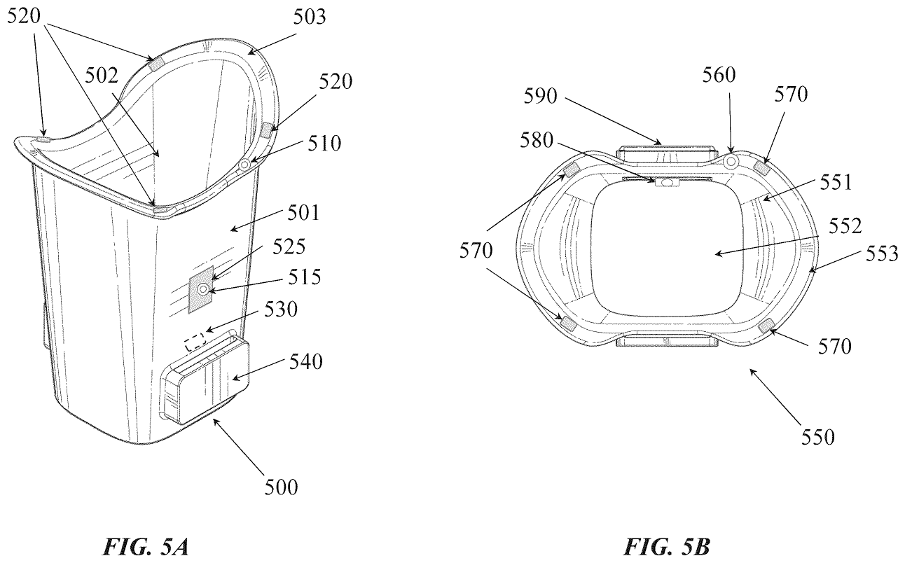

FIG. 5A schematically illustrates an example of a hollow tubular adapter 500. In some cases, the hollow tubular adapter 501 comprises two openings. One opening 552 (FIG. 5B) of the hollow tubular adapter 501 can be attached to the mobile digital camera. A second opening 502 of the hollow tubular adapter 501 can be in contact with the user inner lips. The lips can lay on the outer surface 503 of the second opening 502 of the hollow tubular adapter. On the outer surface 503 of the second opening 502 of the hollow tubular adapter there can be a photoplethysmogram (PPG) sensor 510, and at least one electrode 520. One electrode 525 can be placed on the side of the hollow tubular adapter 501. In some embodiments, a photoplethysmogram (PPG) sensor 515 can be placed on the side of the hollow tubular adapter 501. A flow sensor 530 can be placed on the interior of the hollow tubular adapter 501. In some embodiments, the flow sensor 530 is a turbine. All the sensors can be connected to the connection module 540. The connection module can have Bluetooth or Wi-Fi connectivity.

FIG. 5B schematically illustrates a front view of the same FIG. 5A (500) hollow tubular adapter. The hollow tubular adapter 551 can comprise two openings. One opening 552 of the hollow tubular adapter 551 can be attached to the mobile digital camera. The second opening 502 (FIG. 5A) of the hollow tubular adapter 551 can be in contact with the user's inner lips. The user's lips can lay on the outer 553 of the second opening of the hollow tubular adapter. On the outer surface 553 of the second opening of the hollow tubular adapter, there can be a photoplethysmogram (PPG) sensor 560, and at least one electrode 570. A flow sensor 580 can be placed on the interior of the hollow tubular adapter 551. In some embodiments, the flow sensor 580 is a turbine. All the sensors can be connected to the connection module 590. The connection module can have Bluetooth or Wi-Fi connectivity.

According to some embodiments, the photoplethysmogram (PPG) 510 or 560 sensor faces a user's intraoral soft tissue.

According to some embodiments, at least one electrode 520 or 570 faces a user's intraoral soft tissue.

According to some embodiments, the photoplethysmogram (PPG) sensor and the electrodes are connected to the connection module 540, 590.

According to some embodiments, the connection module 540, 590 comprises Bluetooth or Wi-Fi connectivity.

According to some embodiments, the mobile digital device is a Smartphone.

According to some embodiments, the connection module transfers data from the hollow tubular adapter to a smartphone.

To take a physiometric measurement, the user can place the hollow tubular adapter in the oral cavity. The rim 503, 553 can be in contact with the user's inner lips, The lips can lay on the outer surface 503, 553 of the hollow tubular adapter's second opening. In this manner, the user's intraoral soft tissue (such as gums or inner lips) is facing the photoplethysmogram (PPG) 510 or 560 sensor and at least one of the electrodes 520 or 570. A finger of the user's hand can be touching electrode 525 when holding the hollow tubular adapter. The user can move the adapter side by side in accordance to the requirements to capture a dental scan. The (PPG) sensor can emit light. A radiation sensor can be used to read the returns. An example PPG sensor can be from pulshsensor.com company or SEN-11574 of SparkFun electronics. An example flowmeter can be SFM3000-200 from sensirion inc. an exemplary connection module can be STM20 or MAX3010x of SparkFun electronics

In some embodiments, the photoplethysmogram (PPG) sensor can be used to detect blood volume changes in the microvascular bed of tissue. The PPG sensor can be used to generate measurements of: body temperature, blood pressure, pulse, breathing rate, blood flow, hydration, oxygen saturation, blood sugar, or blood cell volume, or any combination thereof. In some embodiments, a photoplethysmogram (PPG) sensor reading taken with at least two electrodes of the intraoral/skin conductivity can be used to generate electric heart activity measurement such as EKG or ECG. In some embodiments, touching the side plate on the hollow tubular adapter with the finger, can make the EKG or ECG reading more accurate.

While preferred embodiments of the present invention have been shown and described herein, it will be obvious to those skilled in the art that such embodiments are provided by way of example only. It is not intended that the invention be limited by the specific examples provided within the specification. While the invention has been described with reference to the mentioned specification, the descriptions and illustrations of the embodiments herein are not meant to be construed in a limiting sense.

Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations or equivalents. It is intended that the following claims define the scope of the invention and that methods and structures within the scope of these claims and their equivalents be covered thereby.

1. A method of gathering physiometric data of a subject, comprising:
(a) providing a dental scan of an intraoral region of the subject;
(b) analyzing one or more properties of the dental scan; and
(c) generating the physiometric data of the subject based on the one or more properties of the dental scan, wherein the dental scan is acquired using a mobile digital camera and a hollow tubular adapter, wherein a first opening of the hollow tubular adapter is attached to the mobile digital camera, and wherein a second opening of the hollow tubular adapter is configured to contact lips of the subject.

2. The method of claim 1, wherein the mobile digital camera or the hollow tubular adapter comprises a light source configured to emit light in at least one of the infrared (IR), ultraviolet (UV) or visible spectrum.

3. The method of claim 1, wherein the physiometric data comprises body temperature, blood pressure, pulse, breathing rate, breathing volume, blood flow, hydration, oxygen saturation, blood sugar, or blood cell volume of the subject, or any combination thereof.

4. The method of claim 1, wherein the one or more properties comprise a color, an absorbance spectra, a spectral distribution, a size, or a shape of one or more tissues in the intraoral region of the subject.

5. The method of claim 1, wherein the physiometric data is generated during performance of the dental scan.

6. The method of claim 1, wherein the dental scan is provided from a data set.

7. The method of claim 1, wherein the generating in (c) comprises comparing the dental scan to a previous dental scan of the subject.

8. The method of claim 1, wherein the hollow tubular adapter comprises a photoplethysmogram (PPG) sensor.

9. The method of claim 7, wherein the PPG sensor is configured to contact a soft tissue of the intraoral region of the subject.

10. The method of claim 1, wherein the hollow tubular adapter comprises one or more electrodes.

11. A system for gathering physiometric data of a subject, comprising: a processor; a storage element connected to the processor; encoded instructions stored in the storage element, wherein the encoded instructions when implemented by the processor, configure the system to:
(a) provide a dental scan of an intraoral region of the subject;
(b) analyze one or more properties of the dental scan; and
(c) generate the physiometric data of the subject based on the one or more properties of the dental scan, wherein the dental scan is acquired using a mobile digital camera and a hollow tubular adapter, wherein a first opening of the hollow tubular adapter is attached to the mobile digital camera, and wherein a second opening of the hollow tubular adapter is configured to contact lips of the subject.

12. The system of claim 10, wherein the mobile digital camera or the hollow tubular adapter comprises a light source configured to emit light in at least one of the infrared (IR), ultraviolet (UV) or visible spectrum.

13. The system of claim 10, wherein the physiometric data comprises body temperature, blood pressure, pulse, breathing rate, breathing volume, blood flow, hydration, oxygen saturation, blood sugar, or blood cell volume of the subject, or any combination thereof.

14. The system of claim 10, wherein the one or more properties comprise a color, an absorbance spectra, a spectral distribution, a size, or a shape of one or more tissues in the intraoral region of the subject.

15. The system of claim 10, wherein the physiometric data is generated during performance of the dental scan.

16. The system of claim 10, wherein the generating in (c) comprises comparing the dental scan to a previous dental scan of the subject.

17. The system of claim 10, wherein the hollow tubular adapter comprises a photoplethysmogram (PPG) sensor.

18. The system of claim 10, wherein the hollow tubular adapter comprises one or more electrodes.

19. A hollow tubular adapter, comprising: a photoplethysmogram (PPG) sensor; at least two electrodes; and a connection module, wherein a first opening of the hollow tubular adapter is configured to attach to a mobile digital device, and wherein a second opening of the hollow tubular adapter is configured to contact lips of the subject.

20. The hollow tubular adapter of claim 19, wherein the photoplethysmogram (PPG) sensor is configured to face an intraoral soft tissue of the subject.
